(19) United States
(12) Patent Application Publication
(10) Pub. No.: US 2010/0179260 A1
(43) Pub. Date: Jul. 15, 2010

(54) Ionic N-Containing Compounds

(75) Inventors: Stefan Busch, Dusseldorf (DE); Paul Birnbrich, Solingen (DE); Joachim Meyer, Hilden (DE); Ronald Klagge, Erkrath (DE)

Correspondence Address: FOX ROTHSCHILD LLP, 997 Lenox Drive, Bldg. #3, Lawrenceville, NJ, 08648, US

(73) Assignee: COGNIS IP MANAGEMENT GMBH, Düsseldorf (DE)

(21) Appl. No.: 12/665,816

(22) PCT Filed: Jun. 12, 2008
(86) PCT No.: PCT/EP2008/004700

§ 371 Date: Dec. 21, 2009

(30) Foreign Application Priority Data
Jun. 21, 2007 (DE) 102007028714.5

(51) Int. Cl. C08K 5/3445; C07D 233/06

(52) U.S. Cl. 524/106; 548/347.1

(57) ABSTRACT

Ionic N-containing compounds of formula (I), wherein the index n is the number 0 or 1 and R¹ to R⁵ independently are hydrogen, C1-C25 alkyl, C3-C25 cycloalkyl or C6-C25 aryl, wherein the alkyl groups can be saturated or unsaturated, straight-chained or branched, and the cycloalkyl groups can be saturated or unsaturated, with the following stipulations: (1) when n=1, at least one of R¹ to R⁵ must contain 8 to 25 carbon atoms, (2) when n=0, at least one of R¹, R², R³ and R⁵ must contain 8 to 25 carbon atoms, (3) the carbon atoms carrying R² and R³ are linked via a C—C single bond or a C═C double bond, (4) when R¹ to R⁵ are alkyl or cycloalkyl, a hydrogen atom linked to a carbon atom can be replaced with OH or NH₂, or an —O—, —COO— or —NH— group can be inserted between two neighboring carbon atoms linked via a C—C single bond, (5) when R¹ to R⁵ are aryl, a hydrogen atom linked to a carbon atom can be optionally replaced with C1-C12 alkyl or C3-C12 cycloalkyl, with the stipulation that the following applies to the alkyl or cycloalkyl group: optionally a hydrogen atom linked to a carbon atom can be replaced with OH or NH2, or an —O—, —COO— or —NH— group can be inserted between two neighboring carbon atoms linked via a C—C-single bond; said compounds being suitable as temporary surfactants, particularly for the production of aqueous emulsions or dispersions.

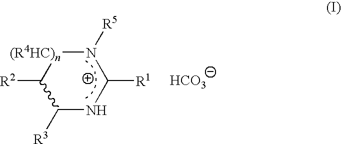

### FIELD OF THE INVENTION

The invention relates to ionic N-containing compounds of specific structure and also to their use as temporary surfactants, especially for preparing aqueous emulsions and dispersions.

### PRIOR ART

P. G. Jessop et al. in Science 2006, Vol. 313, pp. 958-960 describe the reversible reaction of amidines and CO₂ in the presence of water to give the corresponding ionic surfactants. This reaction can be reversed by introduction of an inert gas (nitrogen or argon, for example). Accordingly the authors dub this kind of ionic surfactants switchable surfactants. The activity of such surfactants can be erased as and when required by elimination of CO₂.

Reversible ionic liquids which are liquid at room temperature have been recently described by G. Weiss et al. in Chemistry of Materials 2007, Vol. 19, pp. 967-969. Substances of this kind are occasionally referred to as “green solvents”. The preparation of amidines, however, is not trivial. They can be prepared, for example, from the corresponding dimethyl ketals. These ketals in turn, however, are difficult to access synthetically.

Coalescents (also called film-forming assistants) are known per se. They are added to aqueous coating materials and they bring about the filming of the dispersed polymer particles to form a homogeneous coating film. Their addition is necessary when the film-forming temperature of the binder is above the application temperature.

Known film-forming assistants are as follows: ethylene glycol ethyl ether, ethylene glycol propyl ether, ethylene glycol butyl ether, ethylene glycol hexyl ether, diethylene glycol methyl ether, diethylene glycol ethyl ether, diethylene glycol butyl ether, diethylene glycol hexyl ether, propylene glycol n-butyl ether, dipropylene glycol n-butyl ether, dipropylene glycol methyl ether, tripropylene glycol methyl ether, propylene glycol phenyl ether, propylene glycol tert-butyl ether, 2,2,4-trimethyl-1,3-pentanediol monoiso-butyrate, 2,2,4-trimethyl-1,3-pentanediol diiso-butyrate.

In recent times, water-based coatings have acquired great currency, on environmental grounds. Traditionally, in latex coatings, based in particular on small particles of synthetic polymers such as poly-acrylates, coalescents have been used in substantial quantities. These coalescents (also called filming assistants) are added to the coatings in order to improve film formation. Their function derives from the plasticizing action the coalescent has on the latex particles, enabling these particles to flow together and to form a continuous film. This film has optimum properties after the evaporation of the water. Significant in the context of the formation of a film is the temperature referred to as the film-forming temperature, at which (or below which) the polymer particles flow together to form a film. The customary coalescents lower the film-forming temperature of the polymer.

Conventional coalescents are certain esters and ethers; known technical standards are the hydroxy ester “Texanol” from Eastman (often also called TMB; a 2,2,4-trimethyl-1,3-pentanediol monoisobutyrate) and “EGBE” from Union Carbide (ethylene glycol monobutyl ether).

### DESCRIPTION OF THE INVENTION

In many surfactant applications it is desirable for these surfactants to be able to be “switched off” after they have done their work. What this means is that their character is only temporary and that they can be converted at will into a non-surfactant form.

It was the object of the present invention to provide new temporary surfactants. These surfactants ought to be suitable in particular as emulsifiers and dispersants for emulsion polymerization. The temporary surfactants to be developed ought, moreover, preferably also to possess the property that the nonionic compounds formed when the temporary surfactant is switched off are suitable as coalescents.

A further object of the present invention relates to the preparation of paints and coatings based on aqueous emulsions. Typically, for instance, film-forming latices are prepared by emulsion polymerization. Whereas a suitable surfactant is vital during the emulsion polymerization and the storage of the prepared aqueous emulsion or dispersion up to the time of its use, in order that polymerization takes place to start with and hence the prepared emulsion or dispersion is stabilized, a variety of unwanted quality features of coating films, such as low water resistance, for example, are ascribed to the surfactant that has remained in the coating. A further aspect of the present invention relates to the object of remedying this situation.

Ionic N-containing compounds of the general formula (I),

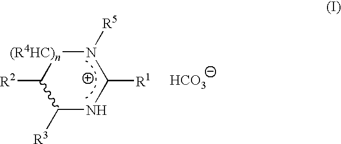

in which the index n is the number 0 or 1 and the radicals R¹ to R⁵ independently of one another are hydrogen or alkyl, cycloalkyl or aryl radicals having 1 to 25 C atoms, it being possible for the alkyl radicals to be saturated or olefinically unsaturated, straight-chain or branched and for the cycloalkyl radicals to be saturated or olefinically unsaturated, with the following provisos: (1) if n is 1, at least one of the radicals R¹ to R⁵ must have 8 to 25 C atoms; (2) if n is 0, at least one of the radicals R¹, R², R³ or R⁵ must have 8 to 25 C atoms; (3) the C atoms carrying the radicals R² and R³ are linked via a C—C single bond or a C═C double bond; (4) in the radicals R¹ to R⁵, where they are alkyl or cycloalkyl radicals, any hydrogen atom joined to a C atom may be substituted by a group —OH or NH₂, or, between two adjacent C atoms linked via a C—C single bond, a group —O—, —COO— or —NH— may be inserted; (5) in the radicals R¹ to R⁵, where they are aryl radicals, any hydrogen atom joined to a C atom may be substituted by an alkyl or cycloalkyl group having 1 to 12 C atoms, with the proviso that for this alkyl or cycloalkyl group the following is true: if desired, a hydrogen atom joined to a C atom can be substituted by a group —OH or NH₂, or between two adjacent C atoms linked via a C—C single bond there may be a group —O—, —COO— or —NH— inserted.

In one embodiment in formula (I) the index n is the number 1. In one embodiment in formula (I) the index n is the number 0 and the C atoms carrying the radicals R² and R³ are linked via a C═C double bond.

In one preferred embodiment in formula (I) the index n is the number 0 and the C atoms carrying the radicals R² and R³ are linked via a C—C single bond. Furthermore, the radical R⁵ has the definition of hydrogen. The compounds characterized thereby have, correspondingly, the formula (I-a)

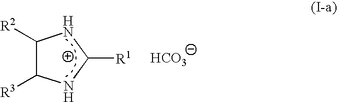

in which the radicals R¹ to R³ independently of one another are hydrogen or alkyl, cycloalkyl or aryl radicals having 1 to 25 C atoms, it being possible for the alkyl radicals to be saturated or olefinically unsaturated, straight-chain or branched, and for the cycloalkyl radicals to be saturated or olefinically unsaturated, with the following provisos: (a) at least one of the radicals R¹, R² or R³ must have 8 to 25 C atoms, (b) in the radicals R¹ to R³, where they are alkyl or cycloalkyl radicals, any hydrogen atom joined to a C atom may be substituted by a group —OH or NH₂, or between two adjacent C atoms linked via a C—C single bond a group —O—, —COO— or —NH— may be inserted, (c) in the radicals R¹ to R³, where they are aryl radicals, any hydrogen atom joined to a C atom may be substituted by an alkyl or cycloalkyl group having 1 to 12 C atoms, with the proviso that for this alkyl or cycloalkyl group the following is true: if desired, a hydrogen atom joined to a C atom may be substituted by a group —OH or NH₂, or between two adjacent C atoms linked via a C—C single bond there may be a group —O—, —COO— or —NH— inserted.

In formula (I-a) the radical R¹ is preferably a saturated or olefinically unsaturated alkyl radical having 8 to 25 C atoms.

In an especially preferred embodiment in formula (I-a) the radical R¹ is a saturated or olefinically unsaturated alkyl radical having 8 to 25 C atoms and the radicals R² and R³ are each hydrogen.

The compounds (I) and (I-a) can be prepared by any of the methods known to the skilled person, as for example by reaction of N-containing compounds of the general formula (II) or (II-a) with CO₂ in the presence of water.

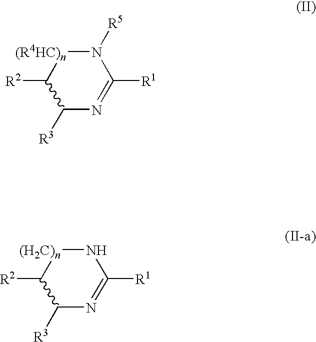

In these formulae (II) and (II-a) the provisos and definitions given above for the formulae (I) and (I-a) apply to the radicals.

The compounds (II) can be prepared by any of the methods known to the skilled person, as for example by reaction of corresponding 1,2-diamines or 1,3-diamines with fatty acids. Examples of suitable 1,2-diamines in this context are ethylenediamine and propylenediamine. A suitable 1,3-diamine is instanced by 1,3-diamino-propane. Examples of suitable fatty acids in this context are the saturated fatty acids hexanoic acid (caproic acid), heptanoic acid, octanoic acid (caprylic acid), nonanoic acid (pelargonic acid), decanoic acid (capric acid), undecanoic acid, dodecanoic acid (lauric acid), tridecanoic acid, tetradecanoic acid (myristic acid), pentadecanoic acid, hexadecanoic acid (palmitic acid), heptadecanoic acid, octadecanoic acid (stearic acid), nonadecanoic acid, eicosanoic acid (arachidic acid), dodecanoic acid (behenic acid), and the olefinically unsaturated fatty acids 10-undecenoic acid, lauroleic acid, myristoleic acid, palmitoleic acid, petroselenic acid, oleic acid, elaidic acid, linoleic acid, linolenic acid, elaostearic acid, gadoleic acid, arachidonic acid, erucic acid, and brassidic acid. It is preferred to use fatty acids of natural origin.

Particularly preferred compounds (II) are characterized by the formula (II-b)

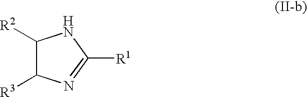

In this formula (II-b) the radicals R¹ to R³ have the same definition as in the formula (I-a) above. The compounds (II-a) represent imidazoline derivatives.

Likewise preferred compounds (II) are characterized by the formula (II-c):

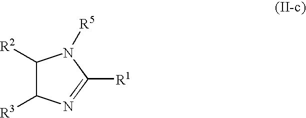

In this formula (II-b) the radicals R¹, R², R³, and R⁵ have the same definition as in the formula (I-a) above. These compounds can be prepared, for example, from N-substituted 1,2-diamines.

A further subject of the invention is the use of the compounds (I) as temporary (switchable) surfactants, more particularly as temporary surfactants for preparing aqueous emulsions and dispersions, and very preferably as temporary surfactants in emulsion polymerization. In this context the switch between the ionic surfactant (I) and the nonionic compounds (II) formed therefrom by elimination of CO₂

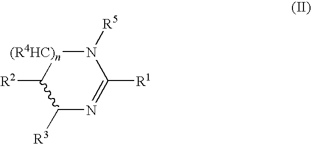

in which the index n is the number 0 or 1 and the radicals R¹ to R⁵ independently of one another are hydrogen or alkyl, cycloalkyl or aryl radicals having 1 to 25 C atoms, it being possible for the alkyl radicals to be saturated or olefinically unsaturated, straight-chain or branched and for the cycloalkyl radicals to be saturated or olefinically unsaturated, with the following provisos: (1) if n is 1, at least one of the radicals R¹ to R⁵ must have 8 to 25 C atoms; (2) if n is 0, at least one of the radicals R¹, R², R³ or R⁵ must have 8 to 25 C atoms; (3) the C atoms carrying the radicals R² and R³ are linked via a C—C single bond or a C═C double bond; (4) in the radicals R¹ to R⁵, where they are alkyl or cycloalkyl radicals, any hydrogen atom joined to a C atom may be substituted by a group —OH or NH₂, or, between two adjacent C atoms linked via a C—C single bond, a group —O—, —COO— or —NH— may be inserted; (5) in the radicals R¹ to R⁵, where they are aryl radicals, any hydrogen atom joined to a C atom may be substituted by an alkyl or cycloalkyl group having 1 to 12 C atoms, with the proviso that for this alkyl or cycloalkyl group the following is true: if desired, a hydrogen atom joined to a C atom will be substituted by a group —OH or NH₂, or between two adjacent C atoms linked via a C—C single bond there may be a group —O—, —COO— or —NH— inserted, is brought about by means of an inert gas, nitrogen or argon for example.

Particularly preferred in this context are the above-defined compounds (1-a) from which elimination of CO₂ produces the above-defined nonionic compounds (II-a).

The compounds (I) for use as temporary surfactants in accordance with the invention can be employed in pure form or in the form of mixtures with one another. If desired the compounds (I) can also be used in combination with other surfactants.

The invention further provides a process for producing coatings based on aqueous polymer dispersions or latex dispersions, characterized in that the aqueous polymer dispersions or latex dispersions are prepared using the above-defined temporary surfactants (I) as emulsifiers, and the temporary surfactants (I), when the aqueous polymer dispersions or latex dispersions are applied, are converted by elimination of CO₂ into the above-defined nonsurfactant compounds (II), which then serve as coalescents.

The term “coalescents” (in the literature also called filming assistants or film-forming assistants) is to be understood in the sense set out above and very well known to the skilled worker.

There are no particular restrictions concerning the nature of the polymer particles or latex particles present in the aqueous dispersions. It is therefore possible to use all of the polymers and copolymers that are relevantly known to the skilled worker for coating purposes.

The aqueous dispersions may, moreover, comprise further adjuvants and additives, known relevantly to the skilled worker, in accordance with the desired end-use application and/or with the nature of the coating.

The aqueous dispersions comprising temporary surfactants (I) for use in accordance with the invention may be applied in principle to any desired surfaces, as for example to wood, metal, plastic, glass, paper, concrete, masonry, and renders.

### EXAMPLES

### Example 1

a) Preparation of the imidazoline: 150 g (2.5 mol) of ethylenediamine were heated in a 4-necked flask with stirrer, dropping funnel, and reflux condenser, and, under reflux, 279 g (1 mol) of oleic acid (Edenor Ti05, Cognis) were metered in over the course of 3 hours. Following distillative removal of the water of reaction (18 g) and of the excess ethylenediamine, the imidazoline was obtained as a pale yellow solid. The yellow coloration was attributable to unremoved ethylenediamine.
b) Preparation of the ionic surfactant: Introduction of CO₂ into an aqueous suspension of this solid for 4 hours gave a colorless suspension whose surface energy was 30 mJ/m².
c) Switching-off of the ionic surfactant: In the course of 4-hour introduction of nitrogen, a 2-phase system was formed, with a clear, aqueous bottom phase topped by a colorless suspension.
d) Reactivation of the ionic surfactant: Renewed introduction of CO₂ for approximately 5 minutes gave the described emulsion again, which remained stable for at least 3 weeks.

The synthesis of an imidazoline described in example 1, and the further reaction of said imidazoline with CO₂ in the presence of water to give the ionic surfactant, are described in the formula scheme below:

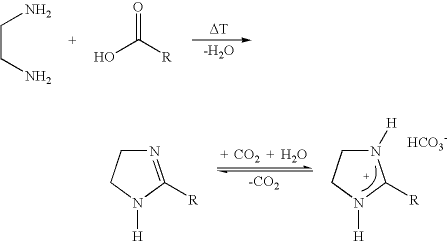

As illustrated by example 1, the ionic surfactant can be considered temporary, since through elimination of CO₂ it can be converted back into the imidazoline (switching-off of the ionic surfactant).

What is claimed is:

1. An ionic nitrogen-containing compound of formula (I), 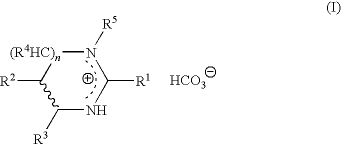
in which the index n is the number 0 or 1 and R¹ to R⁵ independently are hydrogen, C1-C25 alkyl, C3-C25 cycloalkyl or C6-C25 aryl, the acyclic alkyl groups being saturated or unsaturated, straight-chain or branched, and the cycloalkyl groups being saturated or unsaturated, provided that:
(1) when n is 1, at least one of R¹ to R⁵ must have 8 to 25 C atoms;
(2) when n is 0, at least one of R¹, R², R³ and R⁵ must have 8 to 25 C atoms;
(3) the carbon atoms carrying R² and R³ are linked via a C—C single bond or a C═C double bond;
(4) when R¹ to R⁵, are alkyl or cycloalkyl, any hydrogen atom joined to a carbon atom therein may be replaced with OH or NH₂; or, between two adjacent carbon atoms linked via a C—C single bond, a bridging group selected from the group consisting of —O—, —COO— and —NH— may be inserted;
(5) where R¹ to R⁵ are aryl, any hydrogen atom joined to a carbon atom therein may be replaced with C1-C12 alkyl or C3-C12 cycloalkyl, provided that for said alkyl or cycloalkyl substituent the following is true: a hydrogen atom joined to a carbon atom therein may be replaced with OH or NH₂, or between two adjacent carbon atoms linked via a C—C single bond there may be inserted a bridging group selected from the group consisting of —O—, —COO— and —NH— inserted.

2. The compound of claim 1 wherein there is an OH or NH₂ group on the terminal carbon atom of one of R¹ to R⁵.

3. The compound of claim 1 wherein R² to R⁴ are hydrogen, R¹ is C8-C25 alkyl, and R⁵ is hydroxyethyl.

4. The compound of claim 1 wherein said index n is the number 1.

5. The compound of claim 1 wherein said index n is the number 0, and the carbon atoms carrying R² and R³ are linked via a C═C double bond.

6. The compound of claim 1 wherein said index n is the number 0, and the carbon atoms carrying R² and R³ are linked via a C—C single bond.

7. The compound of claim 6 wherein R¹ is a saturated or unsaturated C8-C25 alkyl group.

8. The compound of claim 7 wherein R², R³, and R⁵ are hydrogen.

9. (canceled)

10. A method of preparing aqueous emulsions or dispersions comprising adding at least one compound (I) of claim 1 as a temporary surfactant.

11. A method of emulsion polymerization comprising adding at least one compound (I) of claim 1 as a temporary surfactant.

12. The method of claim 16 wherein the switching between the ionic surfactant (I) and the nonionic compound (II) formed from it by elimination of CO₂, 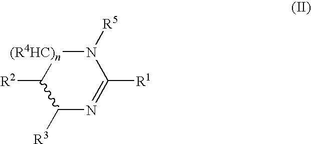
in which the index n is the number 0 or 1 and R¹ to R⁵ independently are hydrogen, C1-C25 alkyl, C3-C25 cycloalkyl or C6-C25 aryl, the acyclic alkyl groups being saturated or unsaturated, straight-chain or branched, and the cycloalkyl groups being saturated or unsaturated, provided that:
(1) when n is 1, at least one of R¹ to R⁵ must have 8 to 25 C atoms;
(2) when n is 0, at least one of R¹, R², R³ and R⁵ must have 8 to 25 C atoms;
(3) the carbon atoms carrying R² and R³ are linked via a C—C single bond or a C═C double bond;
(4) when R¹ to R⁵ are alkyl or cycloalkyl, any hydrogen atom joined to a carbon atom therein may be replaced with OH or NH₂; or, between two adjacent carbon atoms linked via a C—C single bond, a bridging group selected from the group consisting of —O—, —COO— and —NH— may be inserted;
(5) where R¹ to R⁵ are aryl, any hydrogen atom joined to a carbon atom therein may be replaced with C1-C12 alkyl or C3-C12 cycloalkyl, provided that for said alkyl or cycloalkyl substituent the following is true: a hydrogen atom joined to a carbon atom therein may be replaced with OH or NH₂, or between two adjacent carbon atoms linked via a C—C single bond there may be inserted a bridging group selected from the group consisting of —O—, —COO— and —NH— inserted,
is accomplished by means of an inert gas.

13. A process for producing coatings based on an aqueous polymer dispersion or latex dispersion wherein said dispersion is prepared using at least one temporary surfactant (I) of claim 1
as an emulsifier, and wherein said temporary surfactant (I), when said dispersion is applied, is converted by elimination of CO₂ into the nonsurfactant compound (II) of claim 12, which then acts as a coalescent.

14. The process of claim 13 wherein the elimination of CO₂ from said temporary surfactant takes place by means of an inert gas.

15. The process of claim 14 wherein said inert gas comprises nitrogen or argon.

16. A method of providing a temporary (switchable) surfactant comprising adding at least one compound of formula (I) of claim 1.
